(19) United States
(12) Patent Application Publication
(10) Pub. No.: US 2022/0280236 A1
(43) Pub. Date: Sep. 8, 2022

(54) SYSTEM AND METHOD FOR APPLYING CONTROLLED DOSAGE LIGHT THERAPY FOR TREATMENT OF BODY TISSUE

(71) Applicant: RALPH ZIPPER, MELBOURNE, FL (US)

(72) Inventor: RALPH ZIPPER, MELBOURNE, FL (US)

(21) Appl. No.: 17/750,363

(22) Filed: May 22, 2022

Related U.S. Application Data
(63) Continuation-in-part of application No. PCT/US20/61712, filed on Nov. 22, 2020.
Application No. PCT/US20/61712 is a continuation-in-part of application No. 17/746,903, filed on May 17, 2022.
Application No. 17/746,903 is a continuation of application No. 16/171,349, filed on Oct. 25, 2018, now abandoned.
Application No. 16/171,349 is a continuation-in-part of application No. PCT/US17/21273, filed on Mar. 8, 2017.
Application No. PCT/US17/21273 is a continuation-in-part of application No. 15/452,958, filed on Mar. 8, 2017, now abandoned.
Application No. 15/452,958 is a continuation-in-part of application No. 12/687,991, filed on Jan. 15, 2010, now Pat. No. 10,743,929.
Application No. 12/687,991 is a continuation-in-part of application No. 12/496,216, filed on Jul. 1, 2009, now Pat. No. 8,795,264.
Application No. 12/496,216 is a continuation-in-part of application No. 17/732,435, filed on Apr. 28, 2022.
Application No. 17/732,435 is a continuation of application No. 15/452,958, filed on Mar. 8, 2017, now abandoned.
Application No. 15/452,958 is a continuation-in-part of application No. 12/687,991, filed on Jan. 15, 2010, now Pat. No. 10,743,929.
(60) Provisional application No. 62/939,080, filed on Nov. 22, 2019.
(60) Provisional application No. 61/077,348, filed on Jul. 1, 2008.

(51) Int. Cl. A61B 18/24; A61N 5/06; A61B 18/14

(52) U.S. Cl. CPC A61B 18/24; A61N 5/0601; A61B 18/1402; A61B 17/42

(57) ABSTRACT

Methods for improving transmucosal delivery of Photobiomodulation to the body of a patient. Near infrared energy can be delivered through natural body openings in order to deliver therapeutic irradiance to deep tissues and organs. The use of a double sheath mechanism of energy introduction may decrease the discomfort associated with the present method of delivery among those patients with allodynia or severe hyperalgesia. Energy device power settings can be further optimized to improve outcomes and decrease the incidence of known adverse events. Furthermore, the availability of intuitive large screen user interfaces provides a unique opportunity to gather real-time, real-world patient experience (RWE) data.

### CROSS REFERENCE TO RELATED APPLICATIONS AND INCORPORATION BY REFERENCE

This non-provisional patent application is a continuation-in-part of international patent application no. PCT/US20/61712, entitled METHODS AND SYSTEMS FOR TRANSMUCOSAL DELIVERY OF PHOTOBIOMODULATION TO TISSUE, filed in the United States Patent and Trademark Office (USPTO) Receiving Office (USRO) on Nov. 22, 2020, the disclosure of which is incorporated by reference herein in its entirety; PCT/US20-61712 is a non-provisional of, and claims benefit of priority to, U.S. provisional patent application Ser. No. 62/939,080 entitled METHODS AND SYSTEMS FOR TRANSMUCOSAL DELIVERY OF PHOTOBIOMODULATION TO TISSUE, filed in the United States Patent and Trademark Office (USPTO) on Nov. 22, 2019, the disclosure of which is also incorporated by reference herein in its entirety; this application is also a continuation-in-part of U.S. patent application Ser. No. 17/746,903, filed in the USPTO on May 17, 2022, which is a continuation of U.S. patent application Ser. No. 16/171,349, filed in the USPTO on Oct. 25, 2018 entitled SYSTEM AND METHOD FOR APPLYING CONTROLLED DOSAGE LIGHT THERAPY FOR TREATMENT OF BODY TISSUE, which published from the USPTO as U.S. patent application publication number US 2019-0125448 A1 on May 2, 2019, the disclosure of which is incorporated by reference herein in its entirety; U.S. patent application Ser. No. 16/171,349 is a continuation in part (CIP) application of patent application PCT/US17/21273, entitled METHOD FOR TREATING PELVIC PAIN, CHRONIC PROSTATITIS, AND OR OVERACTIVE BLADDER SYMPTOMS, filed in the U.S. Receiving Office under the Patent Cooperation Treaty (PCT) on Mar. 8, 2017, which published as WIPO publication number WO 2018/164676 on Sep. 13, 2018, the disclosure of which is incorporated by reference herein in its entirety; this non provisional patent application is also a continuation-in-part (CIP) application of U.S. patent application Ser. No. 17/732,435, entitled METHOD FOR TREATING PELVIC PAIN, CHRONIC PROSTATITIS, AND OR OVERACTIVE BLADDER SYMPTOMS, filed in the USPTO on Apr. 28, 2022, the disclosure of which is incorporated by reference herein in its entirety; U.S. patent application Ser. No. 17/732,435 is a continuation of U.S. patent application Ser. No. 15/452,958, entitled METHOD FOR TREATING PELVIC PAIN, CHRONIC PROSTATITIS, AND OR OVERACTIVE BLADDER SYMPTOMS, filed in the United States Patent and Trademark Office (USPTO) Mar. 8, 2017, which published as U.S. patent application publication no. US 2017-0172658 A1 on Jun. 22, 2017, the disclosure of which is incorporated by reference herein in its entirety; U.S. patent application Ser. No. 15/452,958 is a continuation-in-part application of U.S. patent application Ser. No. 12/687,991, entitled Bulbous Tipped Surgical Device and Method for Decreasing the Size and/or Changing the Shape of Pelvic Tissues, filed in the USPTO on Jan. 15, 2010, which issued as U.S. Pat. No. 10,743,929 on Aug. 18, 2020, the disclosure of which is also incorporated by reference herein in its entirety; U.S. patent application Ser. No. 12/687,991 was a continuation-in-part of U.S. patent application Ser. No. 12/496,216, entitled METHOD FOR DECREASING THE SIZE AND/OR CHANGING THE SHAPE OF PELVIC TISSUES, filed in the USPTO on Jul. 1, 2009, which issued as U.S. Pat. No. 8,795,264 on Aug. 5, 2014, the disclosure of which is incorporated by reference herein in its entirety; which was a non-provisional application claiming the benefit priority to U.S. provisional patent application Ser. No. 61/077,348, entitled Method for Decreasing the Size and/or Changing the Shape of the Vagina, Labia, Prepuce, Perineum and/or Surrounding Supportive Tissues, filed in the USPTO on Jul. 1, 2008, the disclosure of which is incorporated by reference herein in its entirety; U.S. patent application Ser. No. 16/171,349 is also a continuation-in-part of U.S. patent application Ser. No. 15/452,958; and Ser. No. 16/171,349 is also a continuation-in-part of U.S. patent application Ser. No. 12/687,991. The disclosure of U.S. patent application Ser. No. 12/687,965, entitled Laser Device and Method for Decreasing the Size and/or Changing the Shape of Pelvic Tissues, filed in the USPTO on Jan. 15, 2010, and which published as U.S. patent application publication US 2011-0004203 A1 on Jan. 6, 2011, is also incorporated by reference herein in its entirety.

The disclosures of all patent applications, patent applications, and patents identified above and elsewhere in this application are incorporated herein by reference in their entirety.

### STATEMENT REGARDING FEDERALLY SPONSORED RESEARCH OR DEVELOPMENT

Not applicable.

### INCORPORATION-BY-REFERENCE OF MATERIAL SUBMITTED ON A COMPACT DISK

Not applicable.

### BACKGROUND OF THE INVENTION

### 1. Field of the Invention

The field of the invention relates generally to systems and methods for surgical methods, more specifically, the present invention relates to the transmucosal delivery of energy through a body cavity.

### 2. Background Art

It is estimated that approximately 14% of women age 30 to 50 suffer from Chronic Pelvic Pain (CPP).¹ A recent study of almost 50,000 female U.S. veterans found a 30% incidence of CPP and a 16.8% incidence of opioid use amongst sufferers.² The incidence in of opioid use amongst CPP patients in the general population is consistent with the military cohort.³ Although there are many hypothesized triggers of chronic pelvic pain, a definitive etiology is rarely identified. However, the overwhelming majority of sufferers share a treatable pathology, hypertonicity and tenderness of the pelvic muscles, Levator Myalgia (myofascial pelvic pain) Sixty percent of women with CPP have Levator Myalgia.⁴ Alleviation of this tender pelvic muscle hypertonicity is the mainstay of CPP treatment. Unfortunately, no single therapy is very effective and access to the most effective therapy, skilled manual therapy (PT), is limited.⁵ There is presently no drug indicated for the treatment of chronic pelvic pain. There is presently no medical device cleared for the treatment of pelvic pain. ¹ Mathias, S. D., Kuppermann, M., Liberman, R. F., Lipschutz, R. C., Steege, J. F. Chronic pelvic pain: prevalence, health-related quality of life, and economic correlates. Obstet Gynecol. 1996; 87(3): 321-327.² Cichowski, S. B. et al. A 10-yr Analysis of Chronic Pelvic Pain and Chronic Opioid Therapy in the Women Veteran Population. MILITARY MEDICINE, 00, 0/0:1, 2018³ Adams. K. et al. Levator Myalgia: Why Bother? Int Urogynecol J (2013) 24:1687-1693 and Dr. Charles Buttrick, founder and former president of the International Pelvic Pain Society.⁴ Adams. K. et al. Levator Myalgia: Why Bother? Int Urogynecol J (2013) 24:1687-1693, Kawasaki, A, Amundsen, C. L. OBG Management. Vol. 23. No. 10. October 2011. 17-28, Recognizing Myofascial Pelvic Pain in the Female Patient with Chronic Pelvic Pain, Elizabeth Anne Pastore, P T J Obstet Gynecol Neonatal Nurs. 2012 September; 41(5): 680-691.

Although thousands of published manuscripts and hundreds of randomized controlled trials have described the benefits of Photobiomodulation (PBM) on muscle pain and other painful conditions and Photobiomodulation has been used safely and effectively for decades to treat painful conditions of the human body, it was not until the introduction of SoLá Therapy in 2019 that a method or device became available for the treatment of the human pelvis. The SoLá™ Therapy method and device represent much needed advancements in pelvic medicine. What is needed in the art are improvements in this photobiomodulation apparatus and/or method adapted for the treatment of conditions of the human pelvis.

### BRIEF SUMMARY OF THE INVENTION

The SoLá™ Therapy system (UroShape, LLC) of the invention provides a novel means for transvaginal and/or transrectal delivery of PBM to the muscles and tissues of the pelvis. The invention described and claimed herein provides methods, not previously known or described, for the improvement of transrectal and transvaginal PBM.

The present invention comprises an apparatus and method that have one or more of the following features and/or steps, which alone or in any combination may comprise patentable subject matter.

The present invention overcomes the shortcomings of the prior art in that it provides a means of delivering transvaginal and transrectal PBM without concomitant massage, a method and device needed by those who are in too much pain to tolerate massage; it provides a reproducible method for administering therapeutic irradiance to target pelvic tissues, it provides a method of gathering real world user experience data; it provides device level method of hazard mitigation.

In accordance with one embodiment of the present invention, the invention comprises a system and method for delivering Photobiomodulation to tissue of a patient's body through natural body openings and cavities, wherein the method comprises the steps inserting an outer sheath through the opening and into a body cavity of a patient such as a ⁵ Wein, Alan J Prevalence of Myofascial Chronic Pelvic Pain and the Effectiveness of Pelvic Floor Physical Therapy. The Journal of Urology, Volume 194, Issue 3, 730 vagina or a rectum, secondly inserting a sheath-protected laser optical fiber into the outer sheath, thirdly activating a laser energy source that is meaningfully connected, in other words, optically connected and in optical communication with, the laser optical fiber, next moving said protected laser fiber back and forth inside said tubular sheath, lastly deactivating said laser and removing protected fiber and tubular sheath from said body cavity.

Another aspect of the present invention provides for the use of markings on the tubular sheath or protective sheath of laser optical fiber to input data into laser energy source user interface for optimization of dosing of optical energy to a patient.

An additional aspect of the present invention provides for precise dosing as learned through extensive investigation, collection of real-world experience data, and laboratory evaluation.

An additional aspect of the present invention provides a method in which an laser energy source, or device, user interface is used to mitigate against known procedural hazards.

An additional aspect of the present invention provides a method in which an laser energy source, or device, updates a database located outside the laser energy source (or device) with patient disease state and symptoms information.

An additional aspect of the present invention provides a method in which an laser energy source, or device, gathers symptom data and displays graphical information on its user interface allowing user to visualize symptom improvement or regression

An additional aspect of the present invention provides a method in which an laser energy source, or device, gathers usage data that is communicated to a non-integrated database to maintain procedure related inventory at location of said laser.

In an embodiment, the invention comprises a method for reshaping transmucosal energy delivery to pelvic tissue, said method comprising: a first step of providing an outer sheath with an inner diameter sufficient to allow insertion of a laser fiber probe; followed by a second step of inserting the tubular sheath into a potential space in a patient's body, such as a vagina or rectum; followed by a third step of providing a laser fiber probe composed of a laser optical fiber enclosed in a protective sheath, wherein said laser optical fiber is capable of emitting laser energy from an area in its distal 4 cm, when the optical fiber is meaningfully connected to, or in other words in optical communication with, the source of laser energy; followed by the fourth step of inserting the laser optical fiber probe into the outer sheath until the laser energy emitting portion of the laser optical fiber is located within said body cavity; followed by a fifth step of activating the laser energy source or device and transmitting laser optical energy through the protective sheath and outer sheath; followed by a sixth step of translating the laser optical fiber probe distal end back and forth inside the outer sheath to a distal point not exceeding the distal end of the outer sheath and to a proximal point not exiting the body cavity; followed by a seventh step of deactivating the laser energy source and removing the outer sheath and laser optical fiber probe from body cavity.

In embodiments, the laser energy may comprise laser energy in a wavelength or wavelengths in the range from 400 nm to 1064 nm.

The body cavity may be defined as a vagina or a rectum.

The method of the invention may include the user observing markings on the outer sheath, protective sheath, fiber cladding, or other fiber covering, such markings conveying information on a length of area and or surface area of a patient to be treated. The user may input this observed information into a laser energy source or device user interface for storage and retrieval by a processor in communication with the laser energy source or device, said the observations resulting in determining, alternating or maintaining the laser energy or power dosing level and duration based on dosing information maintained in the laser energy source storage media (memory), or other user input information.

The step of translating may further be defined as moving the laser fiber probe in a distal and proximal motion relative to the site of the second step of insertion.

The step of translating may comprise a time span of at least one minute.

The optical energy output of the laser may be sufficient to deliver an irradiance between 10 and 400 mW/cm2 at a depth 1 cm from the surface of body cavity tissue when the laser fiber probe is inserted into an outer sheath that is disposed in a pelvic cavity in a patient's body.

The optical power output of the source of laser energy source may be set at a power needed to deliver a surface irradiance ranging from desired tissue irradiance at 0.5 mm to 10 mm depth divided by 0.02 to desired tissue irradiance at 0.5 mm to 10 mm depth divided by 0.10 when the laser fiber probe is inserted into an outer sheath that is disposed in a pelvic cavity of a patient's body.

The optical energy output by source of laser energy may be sufficient to deliver an irradiance between 0.050 and 0.150 J/cm2 at a depth 1 cm from the surface of body cavity tissue when the laser optical fiber probe is inserted into an outer sheath that is disposed in a pelvic cavity of a patient's body.

The optical energy output of the laser energy source or device may be between 250 mW and 15 W.

The user may input patient symptom information or diagnostic information into a user interface of the laser energy source, or into a user interface connected to or in communication with the laser every source, prior to activating laser.

The user may input patient symptom information or diagnostic information into user interface of the laser energy source, or into a user interface connected to or in communication with the laser every source, after activating the laser and before treating the next patient.

The invention may generate graphical data to display symptom improvement or worsening.

The invention may transmit patient treatment, laser dosage, laser usage, patient symptom improvement or worsening, or other data to a server or database that is not located within said laser energy source.

The invention may transmit usage information to a server or database that is not located within said laser energy source and such information is used to maintain procedure related inventory at location of the laser.

In embodiments, the invention may comprise a method for using an energy device to treat human tissue, the method comprising a first step of requiring user to respond to a user interface query or queries as part of hazard mitigation prior to allowing activation of said energy device, said hazards having been identified in the device or application failure mode effects analysis, with a second step of allowing activation of laser energy only after a user enters a predetermined response or responses to such query or queries into the user interface of the laser energy source.

In the embodiments of the invention, any of the claimed features may be present, or may not be present, in any combination. Thus, the scope of the claimed invention includes all combinations of the described features and elements.

### BRIEF DESCRIPTION OF THE DRAWINGS

The accompanying drawings, which are incorporated into and form a part of the specification, illustrate one or more embodiments of the present invention and, together with the description, serve to explain the principles of the invention. The drawings are only for the purpose of illustrating the preferred embodiments of the invention and are not to be construed as limiting the invention. In the drawings:

FIG. 1 depicts a break-away side view of an embodiment of an outer sheath of the invention in which the outer sheath is a tube that is open on a first end and closed on a second end.

FIG. 2 depicts a break-away side view of an embodiment of an outer sheath of the invention in which the outer sheath is a tube that is open on a first end and open on a second end.

FIG. 3 depicts a break-away side view of an embodiment of an sheath of the invention in which the protective sheath is a tube that is open on a first end and closed on a second end, and in which the first end has been fluted so as to be of larger diameter than the tube diameter.

FIG. 4 depicts a break-away side view of an embodiment of an outer sheath of the invention in which the outer sheath is a tube that is open on a first end and open on a second end, and in which the first end has been fluted so as to be of larger diameter than the predominating tube diameter.

FIG. 5 depicts an embodiment of the system of the invention in which a laser energy source 005 is connected to an optical laser fiber for transmitting light, i.e. optical, energy from the laser energy source 005 to a light emitting area of the optical fiber 007, and wherein the light emitting area of the optical fiber 007 is disposed within any of the disclosed or structural equivalent embodiments of the outer sheath such that light energy is emitted from the optical fiber, through the wall or walls of the protective fiber sheath and the outer sheath.

### DETAILED DESCRIPTION OF THE INVENTION

The following documentation provides a detailed description of the invention.

Although a detailed description as provided in the attachments contains many specifics for the purposes of illustration, anyone of ordinary skill in the art will appreciate that many variations and alterations to the following details are within the scope of the invention. Accordingly, the following preferred embodiments of the invention are set forth without any loss of generality to, and without imposing limitations upon, the claimed invention. Thus, the scope of the invention should be determined by the appended claims and their legal equivalents, and not merely by the preferred examples or embodiments given.

As used herein, “photobiomodulation” includes within its meaning a form of light therapy that utilizes non-ionizing light sources, including lasers, light emitting diodes, and/or broadband light, in the visible (400-700 nm) and near-infrared (700-1100 nm) electromagnetic spectrum. Photobiomodulation is a nonthermal process involving endogenous chromophores eliciting photophysical (both linear and nonlinear) and photochemical events at various biological scales. This process results in beneficial therapeutic outcomes including but not limited to the alleviation of pain or inflammation, immunomodulation, and promotion of wound healing and tissue regeneration.1 The term photobiomodulation (PBM) therapy is now being used by researchers and practitioners instead of terms such as low level laser therapy (LLLT), cold laser, or laser therapy and thus includes within its meaning the definitions of these terms. There is consensus that the application of a therapeutic dose of light to impaired or dysfunctional tissue, i.e. photobiomodulation, leads to a cellular response mediated by mitochondrial mechanisms that reduce pain and inflammation and speed healing. In embodiments, the primary target (chromophore) for photobiomodulation is the cytochrome c complex which is found in the inner membrane of the cell mitochondria. Cytochrome c is a vital component of the electron transport chain that drives cellular metabolism. As light is absorbed, cytochrome c is stimulated, leading to increased production of adenosine triphosphate (ATP), the molecule that facilitates energy transfer within the cell. In addition to ATP, laser stimulation also produces free nitric oxide and reactive oxygen species. Nitric oxide is a powerful vasodilator and an important cellular signaling molecule involved in many physiological processes. Reactive oxygen species have been shown to affect many important physiological signaling pathways including the inflammatory response. In concert, the production of these signaling molecules has been shown to induce growth factor production, to increase cell proliferation and motility, and to promote extracellular matrix deposition and pro-survival pathways. Outside the cell, nitric oxide signaling drives vasodilation which improves microcirculation in the damaged tissue, delivering oxygen, vital sugars, proteins, and salts while removing wastes.

The scope and breadth of the present inventive disclosure is applicable across a wide variety of procedures, tissues and anatomical structures. Although the following detailed description contains many specifics for the purposes of illustration, anyone of ordinary skill in the art will appreciate that many variations and alterations to the following details are within the scope of the invention. Accordingly, the following preferred embodiments of the invention are set forth without any loss of generality to, and without imposing limitations upon, the claimed invention.

The present method of intracavity transmucosal delivery of photobiomodulation utilizes back and forth movement of bulbous translucent probe. Many patients with pain syndromes cannot tolerate this movement, for any number of reasons, including irritation of the body tissue that is in contact with, or even being displaced by, the bulbous probe as it is translated across pelvic tissue. The improved method of the invention utilizes a two-sheath configuration.

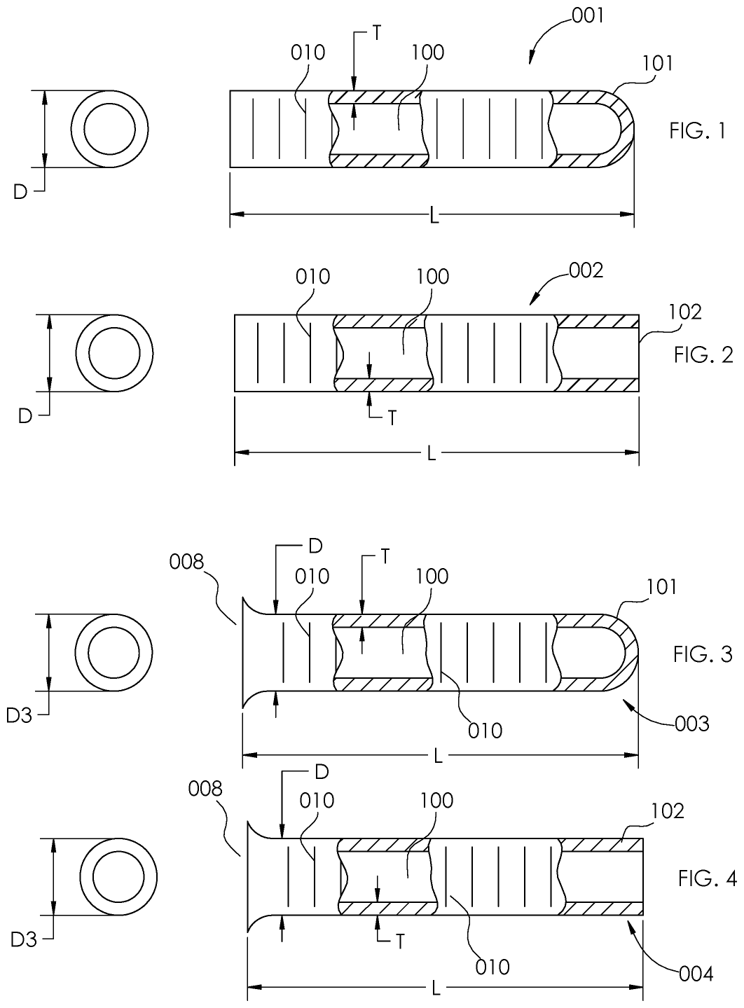

Referring now to FIGS. 1, 2, 3 and 4, an outer sheath 001, 002, 003 or 004, respectively, may comprise material favorable to the transmission of near infrared light. The outer sheath may be inserted into a body cavity such as, by way of example and not limitation, a vagina or rectum of a patient. The outer sheath may be advanced into the body cavity to a maximum depth of proximal treatment (“proximal” being defined as the direction moving away from the medical treatment provider such that the “maximum depth of proximal treatment” is the point of advancement of the proximal end of the outer sheath furthest from the medical provider). In FIGS. 1, 2, 3 and 4, the proximal end is shown as elements 101 (closed end with an arcuate surface) and 102 (open end), but the physical configuration of the proximal end of the outer sheath (e.g. closed, open, partially open or otherwise) may take any physical configuration desired by a user.

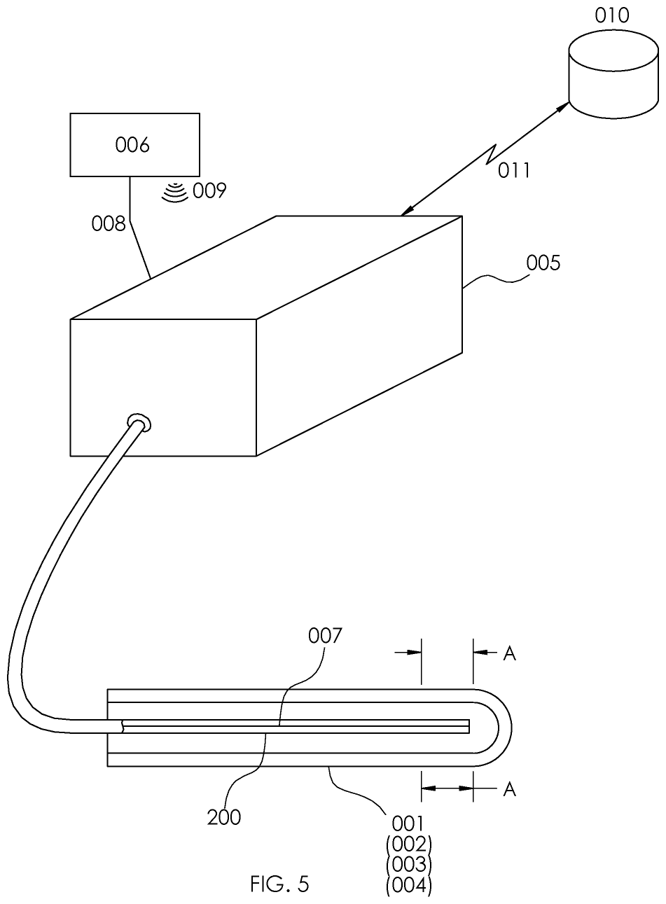

Still referring to FIGS. 1, 2, 3 and 4, the protective sheath 001, 002, 003 or 004, respectively, may be of tubular cross section of outer diameter D, may be of length L, and may have an inner diameter of dimension given by D−2 (T), where T is the tube wall thickness. In embodiments, the tube wall thickness T may be uniform around the outer perimeter of, and along the length L of, the protective sheath 001, 002, 003 or 004. Embodiments may comprise a closed or open first end, 101 and 102, respectively, and may also comprise an optional flared or increased diameter second end 008 as depicted in FIGS. 3 and 4. The optional flared second end 008 may facilitate the insertion of the optical fiber or hand piece 006 structure (not shown in FIGS. 1, 2, 3 and 4 but shown in FIG. 5). The outer sheath 001, 002, 003 or 004 need not be limited to a tubular cross section, nor is it limited to consistent wall thickness along its length or around its periphery. In other words, in embodiments, the outer sheath of the invention may comprise non-uniform wall thickness and may be of non-tubular cross section. The outer sheath may comprise an inner opening 100 into which an optical fiber may be inserted. In a tubular embodiment of the protective sheath, inner opening 100 may be circular in cross section, but such an embodiment is merely exemplary in nature and is therefore non-limiting; i.e., such cross section may be of any shape.

In a preferred embodiment, any embodiment of an outer sheath of the invention, including but not limited to the embodiments depicted as 001, 002, 003 or 004 of the invention may, but do not necessarily, comprise optional markings 010 on an outer surface of the sheath, such as, for example, hash marks placed along the length of the outer sheath, that are used to allow a medical provider to determine the distance between the most proximal treatment point and most distal treatment point. Such optional markings 010 may take any form and may be, but are not necessarily, be of equal spacing between them.

Next, referring now to FIG. 5, the second end of a laser optical fiber probe that is connected to a laser energy source, or device, 005 on its first end is inserted into the outer sheath 001, 002, 003, 004. The laser fiber 007 may transmit laser energy from its distal end in a radial direction, i.e. perpendicular to a longitudinal axis of the optical fiber, for a distance A from its proximal end. In an embodiment, A may be, for example and not by way of limitation, 4 cm. The optical laser fiber 007 may be covered by a protective sheath 200 comprising a material favorable to the transmission of near infrared light. The protective sheath 200 is of an outer diameter smaller than the inner diameter of the outer sheath 001, 002, 003, 004. In an embodiment, the protective sheath 200 of the laser fiber 007 bears markings that are used to determine the distance between the most proximal treatment point and most distal treatment point. In an embodiment, such markings may appear on any non-transmissive protective cladding or other covering that covers the laser fiber 007.

Following insertion of fiber 007 into outer sheath 001, 002, 003, or 004, the fiber, which is in optical communication with and is otherwise meaningfully connected to, or in other words is in optical communication with, a controllable laser power source 005, is activated, meaning that laser energy is delivered from laser source 005 into laser fiber 007. The laser fiber 007 is next motivated back and forth within the outer sheath 001, 002, 003, or 004 such that its most distal end moves in an area between the most distal outer sheath point and a proximal point in the outer sheath 001, 002, 003, or 004 that is within the body cavity. During the movement of laser optical fiber 007 within outer sheath 001, 002, 003, or 004, the outer sheath 001, 002, 003, or 004 may remain stationary with respect to the body cavity body tissue. Following the delivery of a prescribed dose of near infrared energy the laser energy source 005 is deactivated, meaning that optical energy output of the laser is ceased, and the outer sheath 001, 002, 003, or 004 and optical fiber probe 007 and 200 are removed from the body cavity.

In a preferred embodiment, the laser technician visualizes markings on either of the sheaths that provide information on the depth of sheath insertion, which information includes, for example, body cavity length or surface area, which may be used by the invention as described below. Such information may be entered into the laser user interface by a user as input data, which is used as described below. The laser device may comprise or be in communication with a processor that is in communication with computer-readable and writeable physical storage media. The processor may be adapted to read and to store non-transitory computer-readable and computer-executable instructions on said physical storage media, or memory, and to read and to store information on said computer-readable and writeable physical storage media, or memory. The processor may be in communication with various transceivers, wired or wireless, that are in communication with data networks to provide the communications functions described herein, such as, for example, communication with remote servers or databases. The non-transitory computer-readable and computer-executable instructions on said physical storage may be executed by the processor to carry out any or all of the steps and functions of the invention as described herein. The processor may thus write to and maintain on the physical storage media a database of prescribed energy doses based on body cavity length or body cavity surface area, or both, for a specific user. These prescribed energy doses may be determined by the processor, operating on user input data. The entered user input information may be used as input by the processor, and operated upon by the non-transitory computer readable and executable instructions as they are read from the physical storage media and executed by the processor, to determine the dosage appropriate to length of the cavity or surface area of the cavity to be treated, and to control the laser parameters to achieve a desired dosage of laser energy to the body cavity tissue. Prior to applicant's clinical trials, laboratory evaluations, and gathering of real-world experience data, such dosing was not known in the art. Certain body tissues and certain organs may require higher or lower irradiance to achieve photobiomodulation. It is commonly held in the art that higher irradiances negate the beneficial effects of photobiomodulation, however Applicant's results show just the opposite, and were thus unexpected. Further, the prior art was devoid of information regarding the degradation of near infrared energy as it traverses the various chromophores of the pelvic tissues. Whereas proponents of milliwatt laser systems continue to suggest that such low power systems are capable of treating deep tissues, the inventor was surprised to learn that 500 mW was not capable of penetrating transvaginally to the bladder or pelvic muscles at therapeutic irradiance. Such a result was completely unexpected in the art. The inventor was also surprised to find that power loss with depth did not follow a predictable curve. At 4 mm below the surface of the vaginal mucosa 59% of irradiance was lost. At 7 mm depth, 88% of irradiance was lost. At 10 mm depth 97% was lost. These findings have allowed the inventor to define a range of surface powers and deep tissue irradiances that are effective in reducing symptoms of those with myofascial pelvic pain.

Applicant's clinical trials and real-world experience data brought another unexpected result. Patients improved the most with high irradiances at depth, irradiance at the surface of the levator ani muscle neared 100 mW/cm². Applicants were also surprised to learn, and it was not predicted by the prior body of knowledge in the art, that myofascial pelvic pain responded to lower fluences than reported. Excellent results were found at 50-150 mJ/cm². This constellation of discoveries resulted in the inventor discovering the ideal power settings for a photobiomodulation laser treating pelvic tissues transvaginally or transrectally. The power output by laser is set at a power needed to deliver a surface irradiance ranging from desired tissue irradiance at 0.5 mm to 10 mm depth divided by 0.02 to desired tissue irradiance at 0.5 mm to 10 mm depth divided by 0.10. The surface irradiance of any laser may be measured by any laser sensor known in the art. Surface irradiance for a laser is routinely measured during required laser calibration. The preferred embodiment of this method utilizes power settings between 2 and 10 W and wavelengths between 400 and 1064 nm. However other embodiments aimed at treating more superficial structures may need power settings as low as 250 mW and embodiments aimed at treating deeper tissues may need power settings as high as 15 W.

The need for real world experience data in order to improve outcomes and create a more practical regulatory environment has become evident. The preferred embodiment requires the recipient of the photobiomodulation therapy or a technician acting on behalf of the recipient to enter demographic data, and or diagnosis data, and or symptom data, and or other protected health information into the user interface immediately prior to or immediately after each treatment. Such information may be used to generate graphical representations of symptom improvement or exacerbation for viewing on the laser user interface. Such information may also be downloaded by wire, removable drive or wirelessly to a database. Such database may be part of a real-world experience registry (FIG. 3).

In another embodiment of this invention, the laser energy source or device 005 may transmit usage information, such as, for example and not by way of limitation, optical power levels, distance of travel of the laser fiber probe during treatment, duration of treatment and other treatment parameters to a remotely located server or database 010 via an internet connection or other communication means or communication network, which may be any combination of wired or wireless communication means or systems 011, including but not limited to Internet communication through the World Wide Web, where the remote database 010 is not located within said laser energy source or device 005. Such information may be used to maintain procedure-related inventory at a physical location of laser energy source or device 005.

As the most recent update to ISO 14971 no longer allows the Instructions for Use label to reduce risk, device level mitigations are needed. In a preferred embodiment, the laser technician is required to respond to prompts or queries from the laser user interface that are specific to hazards and hazard mitigation identified and or contemplated by the device and or application failure modes effect analysis. In embodiments, only by entering predetermined responses to such queries into the laser interface 006 does the system allow the technician to activate the laser.

While the above description contains much specificity, these should not be construed as limitations on the scope of any embodiment, but as exemplifications of the presently preferred embodiments thereof. Many other ramifications and variations are possible within the teachings of the various embodiments.

In any embodiment, the source of laser energy 005 may be controllable for control of laser energy power output levels, activating (energizing) or de-activating (de-energizing) laser energy, setting predetermined time periods of laser operation, i.e. laser “ON” time during treatment, controlling wavelength of the laser energy, or controlling other laser or laser energy delivery parameters. In any embodiment, the system of the invention may comprise one or more user interfaces 006 that are in communication with the laser energy source 005 via a wireless communication link 009 or a wired connection 008. The user interface 006 may be, for example, any user interface known in the electronic arts that is operable for accepting input commands or data from a user such as, for example and not by way of limitation, one or more keyboards, keypads, mice, touchscreens, wireless input via wireless signal from an electronic device such as a tablet or smart phone upon entry of a command or data into such electronic device by a user, audio input of user voice commands via a microphone, to control any of the above or other laser source parameters, and for accepting input of information from the user as may be required for operation of the functions of the system. The user interface 006 may be in communication with the laser source 005 by wired 008 or wireless 009 communication. Wireless communication link may be, for example any link such as Bluetooth®, WiFi®, or any other radio frequency or optical wireless communication link. User interface 006 may also comprise the ability to display or otherwise convey information to a user, such as, for example and not by way of limitation, one or more video displays or communication links to remote video displays, speakers for communicating audible speech or audio signals of any types such as beeps, tones, or other audio signals.

The scope of the invention should be determined by the appended claims and their legal equivalents, and not by the specific examples given shown in the written description and in the drawings.

What is claimed is:

1. A method for reshaping transmucosal energy delivery to pelvic tissue, said method comprising:
a first step of providing an outer sheath with an inner diameter sufficient to allow insertion of a laser fiber probe;
followed by a second step of inserting said tubular sheath into a patient body cavity potential space such as a vagina or rectum;
followed by a third step of providing a laser fiber probe composed of a laser fiber enclosed in a protective sheath, wherein said laser fiber is capable of emitting laser energy from an area in its distal 4 cm, with said fiber meaningfully connected to a laser energy source;
followed by the fourth step of inserting said laser fiber probe into the outer sheath until the laser emitting portion of said laser fiber is within said body cavity;
followed by a fifth step of activating said laser energy and transmitting said laser energy through said protective sheath and outer sheath;
followed by a sixth step of translating said laser fiber probe distal end back and forth inside the outer sheath to a distal point not exceeding the distal end of the outer sheath and to a proximal point not exiting the body cavity;
followed by a seventh step of deactivating said laser energy and removing said outer sheath and laser fiber probe from body cavity.

2. The method of claim 1, wherein said laser energy comprises laser energy in a wavelength in the range from 400 nm to 1064 nm.

3. The method of claim 2, wherein said body cavity is further defined as a vagina.

4. The method of claim 2, wherein said body cavity is further defined as a rectum.

5. The method of claim 1, wherein the user observes markings on outer sheath, protective sheath, fiber cladding, or other fiber covering such marking conveying information on a length of area and or surface area to be treated, user inputs said observations into a user interface of said laser energy source, said input observations resulting in alteration or maintenance of dosing based on dosing information maintained in laser memory.

6. The method of claim 1, wherein said fourth step of translating is further defined as moving said laser fiber probe in a distal and proximal motion relative to the site of said second step of insertion.

7. The method of claim 1, wherein said fourth step of translating comprises a time span of at least one minute.

8. The method of claim 2 wherein the energy output by laser energy source is sufficient to deliver an irradiance between 10 and 400 mW/cm2 at a depth 1 cm from the surface of body cavity tissue when the laser fiber probe is inserted into an outer sheath that is disposed in a pelvic cavity.

9. The method of claim 2 wherein the power output of said source of laser energy is set at a power needed to deliver a surface irradiance ranging from desired tissue irradiance at 0.5 mm to 10 mm depth divided by 0.02 to desired tissue irradiance at 0.5 mm to 10 mm depth divided by 0.10 when the laser fiber probe is inserted into an outer sheath that is disposed in a pelvic cavity.

10. The method of claim 2 wherein the energy output by source of laser energy is sufficient to deliver an irradiance between 0.050 and 0.150 J/cm2 at a depth of 1 cm from the surface of body cavity tissue when the laser fiber probe is inserted into an outer sheath that is disposed in a pelvic cavity.

11. The method of claim 2 wherein the energy output by laser is between 250 mW and 15 W.

12. The method of claim 2 in which the user inputs patient symptom information or diagnostic information into a laser user interface prior to activating laser.

13. The method of claim 2 in which the user inputs patient symptom information or diagnostic information into the laser user interface after activating said laser and before treating next patient.

14. The method of claim 11 in which laser thence generates graphical data to display symptom improvement or worsening on said user interface.

15. The method of claim 12 in which laser thence generates graphical data to display symptom improvement or worsening on said user interface.

16. The method of claim 11 in which laser thence transmits data to a database that is not located within said laser.

17. The method of claim 12 in which laser thence transmits data to a database that is not located within said laser energy source.

18. The method of claim 1 in which said laser transmits usage information to a database that is not located within said laser and such information is used to maintain procedure related inventory at location of said laser.

19. A method for using an energy device to treat human tissue, said method comprising;
a. a first step of requiring user to respond to a user interface query or queries as part of hazard mitigation prior to allowing activation of said energy device, said hazards having been identified in the device or application failure mode effects analysis; and
b. a second step of allowing activation of laser energy only after a user enters a predetermined response or responses to such query or queries into the user interface.
